(19) United States
(12) Patent Application Publication
(10) Pub. No.: US 2026/0207513 A1
(43) Pub. Date: Jul. 23, 2026

(54) PHARMACEUTICAL COMPOSITION OF SIPONIMOD

(71) Applicant: Synthon B.V., Nijmegen (NL)

(72) Inventors: Luis NOGUEIRAS NIETO, Sant Boi de Llobregat (ES); Lisardo ALVAREZ FERNANDEZ, Sant Boi de Llobregat (ES); Rohit KUMAR, Sant Boi de Llobregat (ES)

(21) Appl. No.: 19/137,775

(22) PCT Filed: Dec. 11, 2023
(86) PCT No.: PCT/EP2023/085195

§ 371 Date: Jun. 11, 2025

(30) Foreign Application Priority Data
Dec. 12, 2022 (EP) 22212934.8

(51) Int. Cl. A61K 9/20; A61K 31/397; A61K 47/02; A61K 47/10; A61K 47/12; A61K 47/14; A61K 47/38

(52) U.S. Cl. CPC A61K 9/2072; A61K 9/2018; A61K 31/397; A61K 47/02; A61K 47/10; A61K 47/12; A61K 47/14; A61K 47/38

(57) ABSTRACT

The present invention relates to pharmaceutical composition comprising siponimod and processes for preparation thereof.

### TECHNICAL FIELD

The invention relates to a pharmaceutical composition comprising siponimod, compound of formula (1), and to a process for preparation thereof;

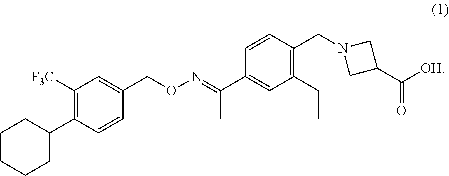 [SMILES: CCc1cc(/C(C)=N/OCc2ccc(C3CCCCC3)c(C(F)(F)F)c2)ccc1CN1CC(C(=O)O)C1]

### BACKGROUND OF THE PRESENT INVENTION

Siponimod, 1-{4-[1-(4-cyclohexyl-3-trifluoromethyl-benzyloxyimino)-ethyl]-2-ethyl-benzyl}-azetidine-3-carboxylic acid, is a sphingosine-1-phosphate (SIP) receptor agonist. Siponimod has been approved for the oral treatment of multiple sclerosis (MS) and is marketed as a tablet under the brand name MAYZENT® (Novartis).

Siponimod was first described in patent application WO2004/103306. Immediate release pharmaceutical compositions comprising siponimod have been described in WO2009/048993, WO2012/093161, and WO2015/155711.

WO2009/048993 discloses compositions comprising SIP receptor modulators; such as 2-substituted 2-aminopropane-1,3-diol or 2-aminopropanol derivatives, which are suitable for use as an oral dosage form. One SIP modulator mentioned is siponimod. No particular example of siponimod composition is disclosed in the application.

WO2012/093161 discloses a solid phase pharmaceutical composition comprising siponimod, wherein the siponimod is not exposed to a basic compound. Due to the non-exposure to basic compounds, the stability of siponimod in the composition can be increased.

WO2015/155711 discloses a dosage form, obtained by a process comprising pre-blending the active pharmacological ingredient with a moisture-protective agent, which provides for stable composition with increased compressibility. The invention relates to the use of agglomerates comprising siponimod and moisture protective agent.

However, there is still a need of alternative and improved formulations that provide for stable pharmaceutical formulations. It is therefore desirable to develop an economical, straightforward, and stable pharmaceutical composition of siponimod which overcome the problems of the prior art and which is advantageously manufactured and is bioequivalent to the commercial siponimod tablet MAYZENT© (Novartis).

### BRIEF DESCRIPTION OF THE INVENTION

The present invention relates to a pharmaceutical composition comprising a mixture of siponimod, compound of formula (1), or solvate, hydrate, or a co-crystal thereof or a salt thereof and one or more hydrated excipients; and to a process for preparation thereof;

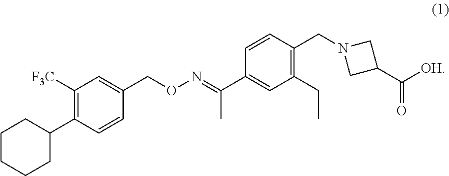 [SMILES: CCc1cc(/C(C)=N/OCc2ccc(C3CCCCC3)c(C(F)(F)F)c2)ccc1CN1CC(C(=O)O)C1]

The pharmaceutical composition of the present invention is simple, stable; it is advantageously manufactured, mimics the dissolution profile of, and is bioequivalent to the commercial siponimod tablet MAYZENT© (Novartis).

### BRIEF DESCRIPTION OF THE DRAWINGS

FIG. 1 depicts the dissolution profiles of composition according to Example 2.

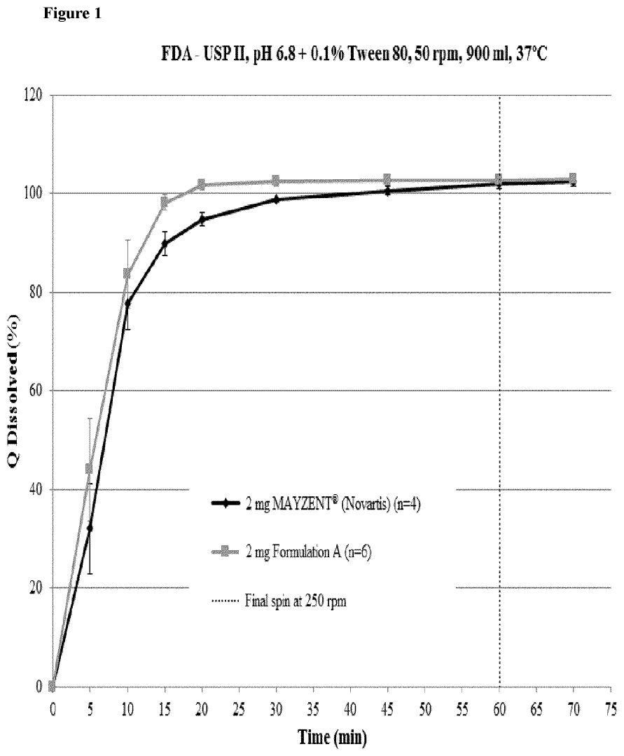

FIG. 2 depicts the process according to Example 2.

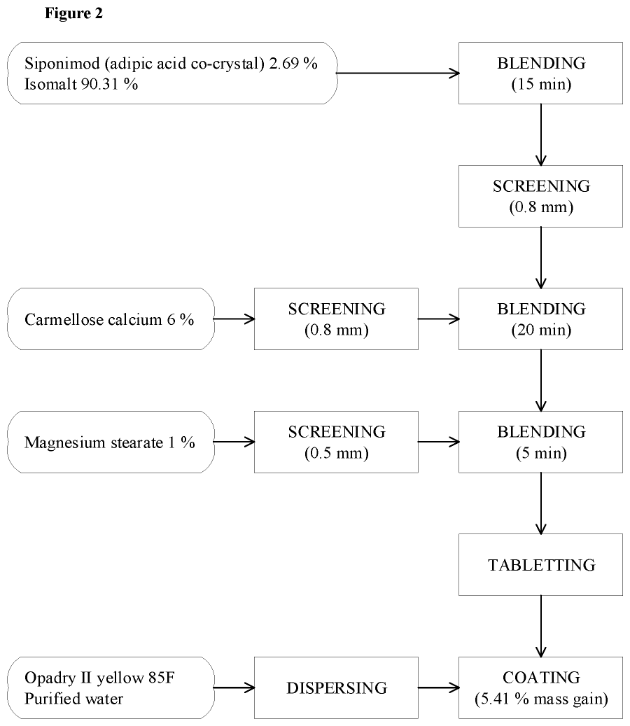

### DETAILED DESCRIPTION OF THE INVENTION

The present invention relates to a pharmaceutical composition comprising a mixture of siponimod or solvate, hydrate, or a co-crystal thereof or a salt thereof and one or more hydrated excipients.

Siponimod was found to be difficult to formulate into a chemically stable solid pharmaceutical composition. There are several means taught in the prior art to stabilize such compositions. In particular, the prior art teaches the avoidance of intimate contact of siponimod with basic compounds. Producing siponimod particles with D(90) diameter of at least 8 μm and crystallinity of 80% or more. Pre-treating the particles with moisture protecting agent. Adding desiccant to such compositions, and hindering ingress of water into such compositions by moisture protecting barrier coating. It is clear the aforementioned teachings render such substantially dry compositions advantageous.

On the contrary, surprisingly, the inventors have found that the presence of water in formulations comprising a mixture of siponimod or solvate, hydrate, or a co-crystal thereof or a salt thereof and one or more hydrated excipients is advantageous, providing for chemically stable compositions without the need for pre-treatment of siponimod or its related compound particles by moisture protecting agent and/or addition of desiccant. Moreover, there is no need to hinder ingress of water into such hydrated compositions by moisture protecting barrier coatings. Thus, pharmaceutical compositions comprising a mixture of siponimod or solvate, hydrate, or a co-crystal thereof or a salt thereof and one or more hydrated excipients of the present invention are simple to prepare, are stable, and mimic the dissolution profile of the commercial siponimod tablet MAYZENT® (Novartis).

In a first embodiment, the present invention relates to a pharmaceutical composition comprising a mixture of siponimod or solvate, hydrate, or a co-crystal thereof or a salt thereof and one or more hydrated excipients, wherein one or more hydrated excipients are present in an amount of 60% to 99.9%, preferably 75% to 99.9%, more preferably 90% to 99.9% by weight based on the total mixture weight.

Particularly, the pharmaceutical composition of the present invention comprises a therapeutically effective dose of siponimod or solvate, hydrate, or a co-crystal thereof or a salt thereof having a particle size distribution D(90) from 3 μm to 100 μm, preferably from 10 μm to 70 μm, more preferred from 30 to 50 μm.

The D(90) value of the particle size distribution is defined as the particle diameter at which 90% by volume of the particles have a smaller diameter than the diameter which corresponds to the D(90) value measured by laser diffractometry. Specifically, a Malvern Instruments Mastersizer was used to determine the particle size distribution.

Further, the present invention provides for a pharmaceutical composition, wherein siponimod or solvate, hydrate, or a co-crystal thereof or a salt thereof is present in an amount of 0.1% to 40%, preferably from 0.1% to 25%, more preferably from 0.1% to 10% by weight based on the total mixture weight.

In a preferred embodiment of the present invention, siponimod or solvate, hydrate, or a co-crystal thereof or a salt thereof is present in an amount of 0.1% to 10% by weight based on the total mixture weight.

Moreover, the present invention provides for a pharmaceutical composition, wherein water is present in an amount of from 5% to 20% by weight based on the total weight of one or more hydrated excipients. The water content can be determined according to the Karl Fischer method as described in Ph. Eur. section 2.5.32. The determination is done by coulometry, preferably Mettler Toledo DL39 Karl Fisher Coulometer, or an equivalent apparatus, including a titration cell without diaphragm. Alternatively, the volumetric Karl Fisher method can be used to determine the water content. Typically, a sample of 250 mg of the hydrated excipient is analyzed. The water content can be achieved by choosing appropriate ingredients, drying, wetting or combination thereof.

In one embodiment, the pharmaceutical composition as described above is manufactured by direct compression process comprising a preparation of the mixture of siponimod or solvate, hydrate, or a co-crystal thereof or a salt thereof and one or more hydrated excipients.

The hydrated excipients to be used in the mixture of siponimod or solvate, hydrate, or a co-crystal thereof or a salt thereof and one or more hydrated excipients in accordance with the present invention can be chosen from, for example, fillers, binders, disintegrants, lubricants and glidants.

Besides one or more pharmaceutically acceptable excipients can be used additionally in accordance with the present invention. Those can be chosen from, for example, fillers, binders, disintegrants, lubricants and glidants.

Fillers are used to increase the bulk volume of a tablet or capsule. By combining a filler with the active pharmaceutical ingredient, the final product is given adequate weight and size to assist in production and handling. The pharmaceutical composition of the present invention preferably comprises at least one filler.

Fillers are preferably used in an amount of from 5% to 97%, more preferably of from 40% to 95%, most preferably of from 75% to 93% by weight based on the total weight of the composition. Suitable examples of fillers in accordance with the present invention include mannitol, sorbitol, lactose, phosphates, cellulose, hydroxypropyl cellulose, starch, pregelatinized starch, modified starch, sucrose, dextrose, dextrates, maltodextrin, xylitol, isomalt, cyclodextrines, calcium phosphate, calcium sulfate and talc.

In a preferred embodiment of the present invention, the fillers are isomalt, lactose or mixtures thereof.

The pharmaceutical composition of the present invention may also comprise one or more disintegrants. Disintegrants are added to a tablet or capsule composition to promote the breakup of the tablet/capsule into smaller fragments in an aqueous environment, thereby increasing the available surface area and promoting a more rapid release of the active pharmaceutical ingredient. Suitable examples of disintegrants to be used in accordance with the present invention include crospovidone, sodium starch glycolate, croscarmellose sodium, carmellose calcium, carboxymethylcellulose calcium, natural starch, pregelatinized starch, sodium starch, microcrystalline cellulose, methylcellulose, croscarmellose, cross-linked sodium carboxymethylcellulose, cross-linked croscarmellose, cross-linked polyvinylpyrrolidone, sodium alginate and gum. Disintegrants are preferably used in an amount of from 1% to 15% by weight, more preferably of from 2% to 10%, even more preferably of from 5% to 10% by weight based on the total weight of the composition.

In a preferred embodiment of the present invention, the disintegrant is carboxymethylcellulose calcium.

The pharmaceutical composition of the present invention may also comprise one or more lubricants. Lubricants are generally used in order to reduce sliding friction. In particular, to decrease the friction at the interface between the blend and the compression machine. Suitable lubricants in accordance with the present invention include magnesium stearate, calcium stearate, stearic acid, glyceryl behenate, hydrogenated vegetable oil, talc and sodium stearyl fumarate. Lubricants are preferably used in an amount of from 0.5% to 5% by weight, more preferably of from 1% to 3%, even more preferably of from 1% to 2% by weight based on the total weight of the composition.

In a preferred embodiment of the present invention, the lubricant is magnesium stearate.

In one embodiment, the present invention provides for the composition comprising the mixture of siponimod or solvate, hydrate, or a co-crystal thereof or a salt thereof and one or more hydrated excipients, wherein siponimod is used in form of solvate, hydrate, or a co-crystal. Preferably, siponimod is used in form of a co-crystal. More preferably, siponimod is used in form of a co-crystal with adipic acid.

The pharmaceutical composition of the present invention may also optionally comprise one or more binders. Binders ensure that tablets and granules can be formed having the desired or required mechanical strength. Binders that are suitable in accordance with the present invention include povidone, low substituted hydroxypropyl cellulose, hydroxypropyl methylcellulose, microcrystalline cellulose, hydroxymethylpropylcellulose, sodium carboxyl methylcellulose, pregelatinized starch, starch, PEG and gelatin. Binders are preferably used in an amount of from 0.5% to 8%, and more preferred 1% to 6% by weight based on the total weight of the composition.

The pharmaceutical composition of the present invention may also optionally comprise one or more glidants. Glidants enhance product flow by reducing interparticulate friction. A suitable example in accordance with the present invention is colloidal silicon dioxide. Glidants are preferably used in an amount of from 0.2% to 10% by weight, more preferably of from 0.2% to 5%, even more preferably of from 0.2% to 2% by weight based on the total weight of the composition.

In a first preferred embodiment, the pharmaceutical composition of the present invention contains the following ingredients, based on the total weight of the composition:
a. Siponimod or solvate, hydrate, or a co-crystal thereof or a salt thereof in an amount of from 0.1% to 25% by weight;
b. One or more fillers in an amount of from 5% to 97% by weight;
c. One or more disintegrants in an amount of from 1% to 15% by weight;
d. One or more lubricants in an amount of from 0.5% to 5% by weight.

In a specific embodiment, siponimod or solvate, hydrate, or a co-crystal thereof or a salt thereof and one or more fillers are used to form a mixture of siponimod or solvate, hydrate, or a co-crystal thereof or a salt thereof and one or more hydrated excipients, while one or more disintegrants and one or more lubricants are used in addition to the mixture.

In a second preferred embodiment, the pharmaceutical composition of the present invention contains the following ingredients, based on the total weight of the composition:
a. Siponimod or solvate, hydrate, or a co-crystal thereof or a salt thereof in an amount of from 0.1% to 25% by weight;
b. One or more fillers in an amount of from 5% to 97% by weight;
c. One or more disintegrants in an amount of from 1% to 15% by weight;
d. One or more lubricants in an amount of from 0.5% to 5% by weight;
e. Optionally, one or more binders in an amount of from 0.5% to 8% by weight;
f. Optionally, one or more glidants in an amount of from 0.2% to 10% by weight.

In a specific embodiment, siponimod or solvate, hydrate, or a co-crystal thereof or a salt thereof and one or more fillers are used to form a mixture of siponimod or solvate, hydrate, or a co-crystal thereof or a salt thereof and one or more hydrated excipients, while one or more disintegrants, one or more lubricants, optionally one or more binders and optionally one or more glidants are used in addition to the mixture.

The compositions of the present invention can be prepared by mixing siponimod or solvate, hydrate, or a co-crystal thereof or a salt thereof with one or more hydrated excipients, followed by mixing of obtained mixture with one or more pharmaceutically acceptable excipients, optionally followed by encapsulation or compression and film-coating, using equipment and methods well-known to the skilled artisan.

In a preferred embodiment, the composition is prepared by direct mixing process and compressed into tablets, which are then film-coated.

In one embodiment of the present invention, the composition is prepared by simple direct mixing process in which siponimod or solvate, hydrate, or a co-crystal thereof or a salt thereof and one or more hydrated excipients are mixed for a period from 7 to 20 minutes, preferably for around 15 minutes. The obtained mixture is sieved and blended with one or more pharmaceutically acceptable excipients for a period from 10 to 25 minutes, preferably for around 20 minutes. Next, the obtained mixture is blended with one or more pharmaceutically acceptable excipients for a period from 2 to 10 minutes, preferably for around 5 minutes. Finally, the obtained mixture is compressed.

In another embodiment of the present invention, the composition is film-coated.

The present invention also relates to a pharmaceutical composition in the form of tablet comprising mixtures as described hereinabove

The pharmaceutical compositions described herein can be made using conventional methods and equipment well known in the art.

The pharmaceutical compositions of the present invention show an in vitro dissolution profile wherein at least 75% of siponimod is released at fifteen minutes when the composition is subjected to a dissolution study in 900 ml of phosphate buffer pH 6.8+0.1% Tween 80 using a USP apparatus II at 50 rpm at 37° C. Preferably, at least 80% of siponimod is released from the pharmaceutical composition at fifteen minutes. The pharmaceutical composition in accordance with the present invention is bioequivalent to the commercially available siponimod tablets MAYZENT® (Novartis).

The present invention is illustrated by the following Examples.

### EXAMPLES

### Example 1

TABLE 1
Compatibility study - closed container (API: excipient 1:10 (w/w);
t = 1 month; T = 40° C.; RH 75%)

| | |
|---|---|
| w (H2O) / % | A^{imp} (1 m) / % |

Active ingredients

| | | |
|---|---|---|
| Siponimod adipic acid co-crystal | 0.2 | 0.09 |
| Siponimod hemifumarate | 0.2 | 0.09 |

Excipients - substantially dry w(H₂O) < 0.5%

| | | |
|---|---|---|
| Sodium stearyl fumarate | 0.4 | 0.08 |
| Glycerol dibehenate | 0.1 | 0.08 |
| Glycerol monostearate | 0.2 | 0.12 |
| Talc | 0.1 | 0.15 |
| Stearyl alcohol | 0.3 | 0.05 |
| Magnesium stearate | 0.2 | 0.16 |

Excipients - dry 0.5% ≤ w(H₂O) < 5%

| | | |
|---|---|---|
| Magnesium aluminometasilicate | 3.9 | 0.57 |
| Calcium silicate | 1.7 | 0.29 |
| Calcium hydrogenphosphate | 0.5 | 0.54 |
| Isomalt | 2.9 | 0.25 |
| Mannitol | 0.6 | 0.49 |
| Microcrystalline cellulose | 3.5 | 0.43 |
| Colloidal silicon dioxide | 1.3 | 1.91 |
| Crospovidone | 2.1 | 0.38 |

Excipients - hydrated w(H₂O) ≥ 5%

| | | |
|---|---|---|
| Lactose | 5.0 | 0.16 |
| Isomalt | 5.0 | 0.14 |
| Carmellose calcium | 9.7 | 0.17 |

Siponimod adipic acid co-crystal (160 mg) and corresponding excipient (1600 mg) were accurately weighed and placed in a glass container. Afterwards, the two components were blended in Turbula® at 72 rpm for 10 minutes. The resulting blend was compacted in the EKO eccentric compression machine in order to obtain a slug. The slug was milled manually, resulting into a milled blend. The milled blend (440 mg) was accurately weighed and placed in glass, opaque vial. The vial was closed and was stored at 40° C./75% RH for 1 month. The milled blend was then analyzed for a sum of area percent levels of impurities (A^{imp}) by HPLC. The HPLC was performed on Acquity UPLC BEH C8, 150×2.1 mm, dₚ=1.7 μm column. The detection with UV light at wavelength of 259 nm was used. The column was eluted, using a gradient method, with aqueous ammonium acetate (10 mM, pH 5.5) and acetonitrile at column temperature of 50° C. for 30 min.

### Example 2

TABLE 2
Formulation A

| Ingredient | Function | m / mg | w / % |
|---|---|---|---|
| Siponimod (adipic acid co-crystal) | Drug substance | 2.283 | 2.69 |
| Isomalt | Filler | 76.767 | 90.31 |
| Carmellose calcium | Disintegrant | 5.1 | 6.00 |
| Magnesium stearate | Lubricant | 0.85 | 1.00 |

| | | |
|---|---|---|
| Total tablet core weight | 85 | 100.00 |
| Opadry II yellow 85F | 4.6 | 5.41 |
| Total coated tablet weight | 89.6 | 105.40 |

Siponimod adipic acid co-crystal and isomalt were adjusted into a bin of a diffusion blender and the mixture was blended for suitable time (e.g. 15 min). The resulting blend was sifted using a screening mill provided with a suitable sieve mesh (e.g. 0.8 mm) to de-agglomerate. The carmellose calcium was sifted through a screening mill provided with a suitable sieve mesh (e.g. 0.8 mm) to de-agglomerate, added together with the previous blend to an adequate bin of a diffusion blender, and blended for a suitable time (e.g. 20 minutes). The magnesium stearate was sifted through a suitable sieve mesh (e.g. 0.5 mm) and blended with the bulk resulting from previous blends into a diffusion blender (e.g. bin blender) for a suitable time (e.g. 5 minutes). The resulting blend was compressed on a rotary tablet press using appropriate punches (e.g. 6 mm round punches). The coating agent and purified water were adjusted into an adequate container and agitated for suitable time (e.g. 1 hour) or until complete homogenization. The tablets are coated with the coating agent suspension until a suitable weight gain (e.g. 5.4% weight gain related to core tablet).

### Example 3

TABLE 3
Stability study of Formulation A in Alu/Alu blister
at 25° C./60% RH

| | Time/month | | | | | |
|---|---|---|---|---|---|---|
| | 1 | 2 | 3 | 6 | 9 | 12 |
| Assay/% | 100.10 | 99.40 | 100.70 | 98.90 | 99.40 | 102.00 |
| Total impurities | | | | | | |
| A^{imp}/% | 0.19 | 0.23 | 0.26 | 0.30 | 0.33 | 0.26 |

The tablets prepared according to Example 2 were packed in Alu/Alu blister. The packed tablets were stored at 25° C./60% RH and analyzed at time points indicated in Table 3 for an assay of siponimod and a sum of area percent levels of impurities (A^{imp}) by HPLC. The HPLC for assay was performed on Acquity UPLC BEH C8, 150×2.1 mm, dₚ=1.7 μm column. The detection with UV light at wavelength of 259 nm was used. The column was eluted, using a gradient method, with aqueous ammonium acetate (10 mM, pH 5.5) and acetonitrile at column temperature of 50° C. for 20 min. The HPLC for A^{imp} was performed according to Example 1.

### Example 4

TABLE 4
Stability study of Formulation A
in HDPE bottle at 25° C. / 60% RH

| Time / month | 3 | 6 | 9 | 12 |
|---|---|---|---|---|
| Assay / % | 100.3 | 99.9 | 97.5 | 98.7 |
| Total impurities | | | | |
| A^{imp} / % | 0.22 | 0.11 | 0.12 | 0.13 |

The tablets prepared according to Example 2 were packed in HDPE bottle. The packed tablets were stored at 25° C./60% RH and analyzed at time points indicated in Table 4 for an assay of siponimod and a sum of area percent levels of impurities (Aᵢₘₚ) by HPLC. The HPLC for assay was performed according to Example 3 and the HPLC for A^{imp} was performed according to Example 1.

1. A pharmaceutical composition comprising a mixture of siponimod or solvate, hydrate, or a co-crystal thereof or a salt thereof and one or more hydrated excipients.

2. The pharmaceutical composition according to claim 1, wherein one or more hydrated excipients are present in an amount of 90% to 99.9% by weight based on the total mixture weight.

3. The pharmaceutical composition according to claim 1, wherein siponimod is present in an amount of from 0.1% to 10% by weight based on the total mixture weight.

4. The pharmaceutical composition according to claim 1, wherein water is present in an amount of from 5% to 20% by weight based on the total weight of one or more hydrated excipients.

5. The pharmaceutical composition according to claim 1, wherein the hydrated excipient is isomalt.

6. The pharmaceutical composition according to claim 1 further comprising a disintegrant and a lubricant.

7. The pharmaceutical composition according to claim 6, wherein the disintegrant is carboxymethylcellulose calcium.

8. The pharmaceutical composition according to claim 6, wherein the lubricant is magnesium stearate.

9. The pharmaceutical composition according to claim 1, wherein siponimod is used in form of a co-crystal.

10. The pharmaceutical composition according to claim 1, wherein siponimod is used in form of a co-crystal with adipic acid.

11. The pharmaceutical composition according to claim 1, wherein siponimod has a particle size distribution D(90) equal to or less than 50 μm.

12. The pharmaceutical composition according to claim 1, wherein siponimod has a particle size distribution D(90) equal to or more than 30 μm.

13. The pharmaceutical composition according to claim 1, wherein siponimod has a particle size distribution D(90) from 30 μm to 50 μm.

14. The pharmaceutical composition according to claim 1, wherein the pharmaceutical composition is in the form of a tablet.

15. The pharmaceutical composition according to claim 14, wherein the pharmaceutical composition is coated.

16. A process to prepare the pharmaceutical composition according to claim 14 comprising blending siponimod and excipients followed by direct compression.
